(19) United States
(12) Patent Application Publication
(10) Pub. No.: US 2007/0202546 A1
(43) Pub. Date: Aug. 30, 2007

(54) Immunoassay Carrier

(75) Inventors: Timothy Stuart Dwyer, Didcot (GB); Simon William Turner, Oxford (GB); Toni Day, Kidlington (GB)

Correspondence Address: CLARK & ELBING LLP, 101 FEDERAL STREET, BOSTON, MA, 02110, US

(73) Assignee: Oxford Immunotec Limited, Abingdon, Oxon (GB)

(21) Appl. No.: 11/632,368

(22) PCT Filed: Jul. 15, 2005
(86) PCT No.: PCT/GB05/02758

§ 371 Date: Feb. 16, 2007

(30) Foreign Application Priority Data
Jul. 15, 2004 (GB) 0415860.6

(51) Int. Cl. G01N 33/567; C12M 3/00

(52) U.S. Cl. 435/7.2; 435/287.2

(57) ABSTRACT

An immunoassay in provided which comprises (a) providing an assay plate having a plurality of recesses, the recesses having solid supports therein having antibody bound thereto; (b) carrying out an assay in one or more of the recesses but not in all of the recesses having antibody bound thereto; (c) subsequently carrying out an assay in one or more of the recesses that were not used in step (b).

The invention relates to assays and to the assay plates and reagents used in such assays. The invention relates in particular to microtiter plates or other assay plates having antibodies bound thereto and their use and re-use in assays such as ELISPOT assays.

### BACKGROUND TO THE INVENTION

The filter immunoplaque assay, otherwise called the enzyme-linked immunospot assay (ELISPOT), was initially developed to detect and quantitate individual antibody-secreting B cells. At the time it was developed, the technique provided a rapid and versatile alternative to conventional plaque-forming cell assays. Recent modifications have improved the sensitivity of the ELISPOT such that cells producing as few as 100 molecules of a specific protein per second can be detected. These assays take advantage of the relatively high concentration of a given proteinaceous cell product (such as a cytokine) in the environment immediately surrounding the protein-secreting cell. These cell products are captured and detected using high-affinity antibodies. The ELISPOT assay is reviewed in Current Protocols in Immunology, Unit 6. 19 pages 6.19. 1-8.

The ELISPOT assay involves six specific steps: (1) coating a purified cytokine-specific antibody to a membrane-backed microtiter plate; (2) blocking the plate to prevent non-specific absorption of any other proteins; (3) incubating the cytokine-secreting cells with appropriate reagents; (4) removal of cells and reagents; (5) adding a labelled second anti-cytokine antibody; and (6) detecting the antibody-cytokine complex on the membrane.

The ELISPOT assay utilises two high-affinity cytokine-specific antibodies directed against different epitopes on the same cytokine molecule: either two monoclonal antibodies or a combination of one monoclonal antibody and one polyvalent antiserum. ELISPOT generates spots based on a colorimetric reaction that detects the cytokine secreted by a single cell. The spot represents a “footprint” of the original cytokine-producing cell. Spots are permanent and can be quantified visually, microscopically or electronically. Detection methods using fluorescence labels are also practised in the art.

Techniques are well established (Catt and Tregear (1967); Salmon et al. (1969) and Perlmann (1972)) to facilitate the coating of monoclonal or polyclonal antibodies on to PVDF membranes or other solid phases. Key variables include pre-activation of the solid phase, concentration of the antibody solution, pH and ionic strength of the coating buffer, coating time and temperature, and post-coating treatments. All these variables require optimisation in order to provide coated microtiter plates with a high concentration of coated antibody which is evenly distributed on the surface of the solid phase. Pre-coating of the solid phase in microtiter plates for use in ELISPOT assays confers consistent sensitivity and stability across a large number of plates. This is important for routine processing of diagnostic samples.

The ELISPOT assay can be used in a clinical setting, where for example, each kit is able to assay 24 patient samples at one time (using 4 wells per sample in a 96 well plate). A method of recycling the plates and reagents would be a great advantage to the low volume user. Many clinics or laboratories may be unable to obtain 24 fresh blood samples at one time point, for example because of the small number of patients attending a particular clinic session; or some laboratories may prefer to process samples in smaller numbers because of logistical issues, such as availability of personnel and equipment. In either case the option to assay less than 24 samples and to re-cycle or re-use the plate and reagents at a later time would greatly improve laboratory efficiency.

### SUMMARY OF THE INVENTION

In this invention, it has surprisingly been found that the antibody-pre-coated assay plates and reagents can be recycled; that is, the assay can be performed on a portion (a proportion of the total wells) of a microtiter plate, and the plate and reagents can subsequently be stored prior to further assays being performed on another portion of the same plate using additional assay reagents.

In accordance with the present invention, there is provided an immunoassay comprising:
(a) providing an assay plate having a plurality of recesses, the recesses having solid supports therein having antibody bound thereto;
(b) carrying out an assay in one or more of the recesses but not in all of the recesses having antibody bound thereto;
(c) subsequently carrying out an assay in one or more of the recesses that were not used in step (b).

### DESCRIPTION OF THE FIGURES

FIG. 1. Effect of Multi-use test on an ELISPOT plate sealed with an Acetate sealing strip, based on results in Table 1. Graph represents mean +/−1 Standard Deviation.

FIG. 2. Effect of Multi-use test (Long routine) on an ELISPOT plate with an Acetate sealing strip, based on results in Table 2. Graph represents mean +/−1 Standard Deviation.

FIG. 3. Spot counts of Multi-use reagents stored at two temperatures versus control reagents, based on results in Table 3. Graph represents mean +/−1 Standard Deviation. The spot counts for the fresh positive give an artificially low reading due to the well being completely saturated.

FIG. 4. Effect of Multi-use secondary conjugate on ELISPOT plate spiked with varying concentrations of Interferon Gamma, based on results in Table 4. Graph represents mean % saturation +/−1 Standard Deviation. Using a Student T-Test there is no significant difference (α=0.05) between the results obtained at all points.

FIG. 5. Mean±standard deviation SFC counts for T cell lines D454 E12 with Peptide pool 2 in short term study (n=12).

FIG. 6. Mean±standard deviation SFC counts for T cell line D481 B9 with Peptide pool 2 in long term study (n=8).

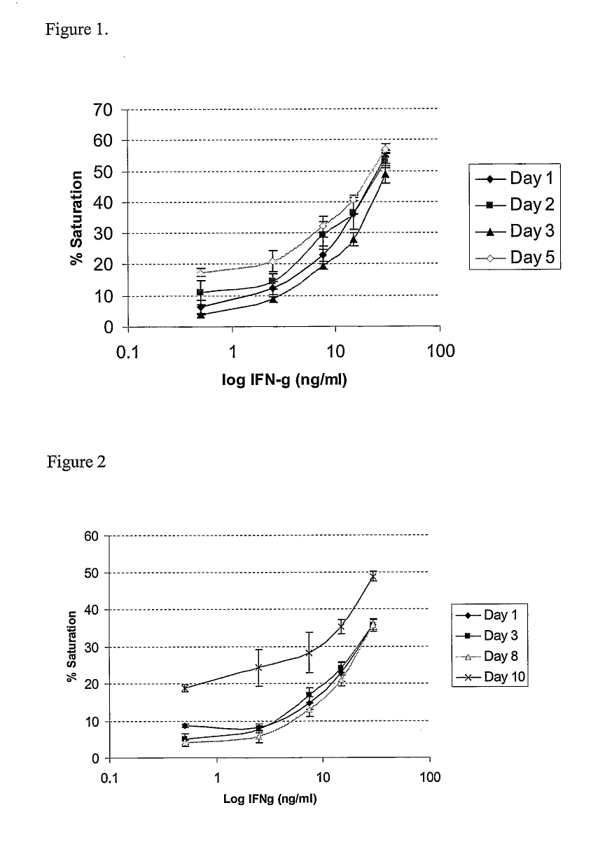

### DETAILED DESCRIPTION OF THE INVENTION

The invention relates to assays using an assay plate having a plurality of recesses or reaction wells, each recess or reaction well having a solid support having antibody bound thereto. The bound antibodies are used in an assay in one or more of the recesses or reaction wells. Such assays may include addition of cells and reagents, and incubation at suitable temperatures. Recesses or reaction wells not used in the first assay can then subsequently be used in an assay carried out at a later time. Several separate assays at separate time points can be carried out using a single plate.

In a preferred aspect of the invention, the assay plate comprises a microtiter plate. Such microtiter plates are widely used. These plates commonly have either 96 or 384 wells. Typically, the wells are arranged in an array, for example 8×12 for a 96 well plate or 24×16 for a 384 well plate. The plates have a plurality of recesses or reaction wells which are designed to be used with samples of approximately 125 μl for the 96 well plate or 30 μl for the 384 well plate. The wells are generally closely spaced in a regular array. Such microtiter plates are typically made of a suitable plastics materials such as polypropylene.

The assay plates of the present invention, such as a microtiter plate may typically be made of a suitable plastics material. The recesses or reaction wells have a solid support having antibody bound thereto. Preferably the solid support is a solid permeable support. Such a solid support is typically a membrane such as a nitrocellulose or PVDF membrane in the bottom of the recess. Alternatively the solid support may comprise a solid base of the recess, such as a solid polystyrene base or one that has been treated to accept or enhance antibody binding.

The solid supports have antibody bound thereto. The antibody is selected depending on the assay to be carried out. Preferably the assays are cell-based assays, and may be an immunospot assay, such as an ELISPOT assay. Typically, assays such as ELISPOT assays are carried out using anti-cytokine antibodies such as antibodies to human interferon gamma, IL-2 or TNF-α. The antibodies are bound to the solid supports by any suitable means. Typically, an assay plate is provided in which the solid supports in each recess or reaction has the same antibody thereto.

Antibodies are immunoglobulin molecules, and are well known in the art (Immunology, Ivan Roitt, et al., Gower Medical Publishing, 1985). They generally comprise two ‘heavy’ and two ‘light’ polypeptide chains linked by sulphydryl bonds, and species that bind to specific antigens with high affinity can routinely be generated as monoclonal or polyclonal species. Typical anti-cytokine antibodies can be produced that are specific for cytokine molecules, such as gamma anti-interferon, IL-2, IL-10 and anti-TNF-alpha. Antibody fragments can also be generated, for example, F(ab)2 fragments. In principle, as long as the molecule retains its antigen-specific binding site, so that it can bind to its corresponding hapten/antigen, then these molecules, including those directed at non-cytokine haptens/antigens, for example steroids, and other protein and non-protein hormones, can be usefully bound to solid phases and used in immunoassays of the type described herein.

Any suitable assay can be carried out with suitable assay reagents being added. The assays may involve incubating the plates at a suitable temperature in accordance with the assay protocol. Typically, such assays incorporate an incubation step between 20 and 60° C., for between 1 and 48 hours. For example, the incubation temperature is typically between 25 and 42° C., such as about 37° C. The incubation step is carried out between 2 and 24 hours, for example for at least 4 hours or at least 8 hours, such as 8 to 12 hours or for at least 12 hours or at least 16 hours such as 16 to 20 hours. Each assay carried out using the same assay plate may be the same or different. Typically, the assays are cell based assays, involving incubation of a sample containing cells in the assay plate.

Recesses or reaction wells which are not being used in any particular assay may be covered during the course of the assay. In one aspect of the present invention, such recesses or reaction wells are sealed. The recesses may be sealed by the use of an adhesive backed film or foil or by heat sealing such a film to the plate surface. Such plate sealers are well known in the art and may comprise acetate membranes or Mylar plate sealers. The entire assay plate may be provided with such a cover prior to the first assay.

The cover is removed from those recesses in which an assay is to be carried out, while the remaining recesses may remain covered or sealed. The cover may be pierced, for example with a pipette in order to allow reagents to be added to the reaction well(s) to be used. The cover over the reaction well(s) to be used may also be completely removed. One or more of the recesses that remain sealed during the first assay can be uncovered in order to carry out the next assay using the assay plate. The cover may be removed from all recesses or reaction wells and re-applied to only cover or seal those recesses that have not yet been used or that are not needed for the next assay. In this way, 10 or more separate assays may be carried out using the same plate. Typically, the same plate is used for 4 to 5 separate assays, the required number of recesses being uncovered prior to each assay. The same or a different number of recesses or reaction wells may be used in each assay.

The plates having antibody bound thereto can be stored between the various assays. Plates can be stored up to 1 year, for example up to 3 to 6 months, or for example up to 40 days, for example up to 10 days between assays, typically for 1 to 2 days between each assay. Preferably, the plates are used for up to 5 separate assays, over a period of 1 year, for example 2 months or 40 days, preferably over a period of 5 to 10 days. The plates can be stored at any suitable temperature, typically between −5 to 10° C., preferably, between 2 to 8° C.

The assay plate may have any suitable number of recesses. Preferably, the assay plate is a microtiter plate having 96 recesses or wells therein. Typically, all of the wells are pre-coated with antibody. The same or a different number of recesses or wells may be used each time that the plate is used in an assay.

Preferably, the plates are used in an ELISPOT assay, wherein the antibody-precoated plates are blocked to prevent non-specific absorption of proteins, the wells are incubated with cytokine secreting cells and appropriate reagents, cells and reagents are removed by washing, labelled second anti-cytokine antibody is added and antibody-cytokine complex is detected. Thus, conjugate reagent containing a second antibody directed to the cytokine antibodies and substrate reagent containing colorometric developing reagents can be used in the assay.

The use and reuse of microtiter plates in such assays allows for a more cost-effective use of antibody-coated plates. Assays may be carried out on a small number of samples without the need to discard a partly used assay plate, the unused wells being used for assaying samples collected at later time points.

The following examples illustrate the invention:

### EXAMPLE 1

### Pre-Coating of PVDF or Nitrocellulose Microtiter Plate Wells for Enzyme Linked Detection

The method for pre-coating antibodies to microtiter plate wells is well defined and common place. Primary antibody levels of 0.01-15 μg per well can be prepared in 50-100 mM carbonate buffer (pH 9.0), added to the desired wells (50-100 μl/well) of a microtiter plate (eg. Multiscreen HTS, Cat. No. MSIPS45, Millipore Corp., Bedford, Mass., USA) and incubated at 4° C. overnight. The coating solution is removed by washing in PBS. These methods are described in Catt and Tregear (1967); Salmon et al. (1969) and Perlmann (1972).

### EXAMPLE 2

### A Typical ELISPOT Assay: Assessment of CEF Peptide Pool

Subjects were healthy laboratory personnel.

CEF peptide pool was obtained from Mabtech, Stockholm, Sweden.

Peripheral Blood Mononuclear Cells (PBMC) were recovered using the Vacutainer CPT system (Becton Dickinson, USA), referring to manufacturers instructions, washed by centrifugation, resuspended in AIM-V™ media (GIBCO™), and added to a 96 well PVDF-backed plate pre-coated with anti-IFNγ Mab 1-DIK (h-IFN-γ ELISpot^{PRO} kit, Mabtech, Stockholm, Sweden) in a final volume of 100 μl AIM-V/well. Input cell numbers were usually 2.5×10⁵ per well. 50 μl of a solution of 2 μg CEF peptides/ml were added. Assays were usually incubated for 16-20 hours at 37° C. in an atmosphere containing 5% CO₂. Incubation was arrested by removing the contents of the wells and washing. 50 μl of 1:200 dilution of Conjugate Reagent (mAb 7-B6-1, h-IFN-γ ELISpot^{PRO} kit, Mabtech, Stockholm, Sweden) was added to all wells and incubated for one hour at room temperature. The wells were again washed and 50 μl of Substrate Reagent (BCIP/NBT, h-IFN-γ ELISpot^{PRO} kit, Mabtech, Stockholm, Sweden) was added. After a further 5-15 minutes the wells were washed with water to terminate the colourimetric reaction. The spots were counted under a magnifying glass and Spot Forming Cells (SFCs) were enumerated.

Control wells contained PBMC in the absence of CEF peptides.

Results:

All members of laboratory staff tested gave a positive response to the peptides.

### EXAMPLE 3

### Recycling of Pre-Coated Microtiter Plates for Use in an ELISPOT Assay

1. Cycling Plates Four Times Over a Period of 5 Days.

Pre-coated 96-well Microtiter Plates (h-IFN-γ ELISpot^{PRO} kit, Mabtech, Stockholm, Sweden) were sealed using Acetate Sealing Strips (ICN Biomedicals, USA). At time zero, 24 wells were exposed by the removal of the Acetate strip. Five solutions of human interferon gamma (Autogen Bioclear) were prepared in AIM-V media (GIBCO)+0.5% Bovine Serum Albumin (BSA) to give final concentrations of 30, 15, 7.5, 2.5 and 0.5 ng/mL. 100 μl/well of each of the solutions was added to the microtiter plate in quadruplicate. AIM-V media alone was used as a negative control. The plates were then incubated at room temperature for one hour. The plates were washed with PBS (GIBCO) and the working strength Conjugate Reagent was added at a volume of 50 μl/well.

The plates were incubated for one hour at room temperature then washed in PBS and 50 μl of Substrate Reagent was added to all wells. After a seven minute incubation at room temperature the substrate was removed and the plate washed in deionised water to stop the reaction.

The plates were allowed to dry for one hour at room temperature and the percentage saturation of each well was analysed using an automated plate reader (AID, Straβberg, Germany) After reading, the plates were placed at 37° C. in a humidified incubator with a 5% CO₂ supply overnight to simulate the incubation conditions of an ELISPOT assay.

On day two, the plates were removed and placed at 2-8° C. for two hours. The next 24 wells were exposed and the assay described above was repeated. After drying, the plates were replaced at 37° C. in a humidified incubator with a 5% CO₂ supply overnight to simulate the conditions of an ELISPOT assay.

On day three the procedure for day two was followed. After incubation at 37° C. the plates were placed in 2-8° C. storage until day five.

On day five the procedure for day two was repeated. The plates were left to dry at room temperature overnight and read on an automated reader.

The control plate was also cycled using the procedure above. This plate was left unsealed as a control.

TABLE 1
Effect of Multi-use test on an ELISPOT plate sealed with an Acetate sealing strip.
Results:

| Day 1 | | | | Day 2 | | | | Day 3 | | | | Day 5 | | | |
|---|---|---|---|---|---|---|---|---|---|---|---|---|---|---|---|
| | Mean | SD | % CV | | Mean | SD | % CV | | Mean | SD | % CV | | Mean | SD | % CV |
| 30 | 54.5 | 2.12 | 3.89 | 30 | 53.25 | 2.50 | 4.69 | 30 | 48.75 | 2.87 | 5.89 | 30 | 57 | 1.63 | 2.86 |
| 15 | 36 | 0.00 | 0.00 | 15 | 36.5 | 5.45 | 14.92 | 15 | 28 | 2.16 | 7.72 | 15 | 40.75 | 0.50 | 1.23 |
| 7.5 | 23 | 2.83 | 12.30 | 7.5 | 29.25 | 4.35 | 14.87 | 7.5 | 19.5 | 1.29 | 6.62 | 7.5 | 32 | 3.37 | 10.52 |
| 2.5 | 12.5 | 2.12 | 16.97 | 2.5 | 14.5 | 2.52 | 17.36 | 2.5 | 8.75 | 0.96 | 10.94 | 2.5 | 21 | 3.46 | 16.50 |
| 0.5 | 6.5 | 2.12 | 32.64 | 0.5 | 11 | 3.92 | 35.60 | 0.5 | 4 | 0.00 | 0.00 | 0.5 | 17.5 | 1.29 | 7.38 |
| 0 | 6.5 | 2.12 | 32.64 | 0 | 8.25 | 2.87 | 34.82 | 0 | 4 | 1.41 | 35.36 | 0 | 14.75 | 2.87 | 19.47 |

Column one shows the concentration of Interferon Gamma used (ng/ml). n = 4. Mean represents % saturation of test wells.

2. Cycling Plates Four Times Over a Period of 10 Days.

Pre-coated 96-well Microtiter Plates were sealed using Acetate Sealing Strips. At time zero 24 wells were exposed by the removal of the Acetate strips. Five solutions of human interferon gamma (Autogen Bioclear) were prepared in AIM-V media (GIBCO)+0.5% Bovine Serum Albumin (BSA) to give final concentrations of 30, 15, 7.5, 2.5 and 0.5 ng/mL. 100 μl/well of the solutions was added to the microtiter plate in quadruplicate. AIM-V media alone was used as a negative control.

The plates were then incubated at room temperature for one hour. The plates were washed with PBS (GIBCO) and the working strength Conjugate Reagent was added at a volume of 50 μl/well.

The plates were incubated for one hour at room temperature then washed in PBS and 50 μl of Substrate Reagent was added to all wells. After seven minutes incubation at room temperature the Substrate Reagent was removed and the plate washed in deionised water to stop the reaction.

The plates were allowed to dry for one hour at room temperature and the percentage saturation of each well was analysed using an automated plate reader. After reading, the plates were placed at 37° C. in a humidified incubator with a 5% CO₂ supply overnight to simulate the conditions of an ELISPOT assay.

On day two the assay plates were placed at 2-8° C. until day three.

On day three the procedure for day one was followed using the next 24 wells on the plate.

On day four the plates were placed at 2-8° C. until day eight.

On day eight the assay was run as described on day one.

On day nine the plates were placed at 2-8° C. until day ten and on day ten the final 24 wells were used to run the assay described on day one. The plates were left to dry overnight at room temperature and then read using an automated reader.

TABLE 2
Effect of Multi-use test (Long routine) on an ELISPOT plate sealed with an Acetate sealing strip.
Results

| Day 1 | | | | Day 3 | | | | Day 8 | | | | Day 10 | | | |
|---|---|---|---|---|---|---|---|---|---|---|---|---|---|---|---|
| | Mean | SD | % CV | | Mean | SD | % CV | | Mean | SD | % CV | | Mean | SD | % CV |
| 30 | 35.75 | 1.26 | 3.52 | 30 | 35.75 | 1.71 | 4.78 | 30 | 35.75 | 1.26 | 3.52 | 30 | 48.75 | 1.26 | 2.58 |
| 15 | 23 | 2.45 | 10.65 | 15 | 24 | 1.83 | 7.61 | 15 | 21 | 1.83 | 8.69 | 15 | 35.25 | 1.89 | 5.37 |
| 7.5 | 14.75 | 1.71 | 11.58 | 7.5 | 16.75 | 2.06 | 12.31 | 7.5 | 13 | 1.83 | 14.04 | 7.5 | 28.25 | 5.38 | 19.04 |
| 2.5 | 8.25 | 0.96 | 11.61 | 2.5 | 7.75 | 0.96 | 12.35 | 2.5 | 5.75 | 1.71 | 29.70 | 2.5 | 24.25 | 4.99 | 20.58 |
| 0.5 | 8.75 | 0.50 | 5.71 | 0.5 | 5 | 1.41 | 28.28 | 0.5 | 4 | 0.82 | 20.41 | 0.5 | 18.75 | 0.96 | 5.11 |
| 0 | 5.75 | 0.96 | 16.65 | 0 | 5.5 | 1.00 | 18.18 | 0 | 3.5 | 0.58 | 16.50 | 0 | 22.25 | 4.99 | 22.43 |

Column one shows the concentration of Interferon Gamma used (ng/ml). n = 4. Mean represents % saturation of test wells

The same experiment was repeated using Mylar plate sealing strips.

The use of plate sealers (Acetate or Mylar) has no detrimental effect on the assay. The results gained from the unsealed control plates are comparable to those gained from the pre-sealed plates, and no contamination or adverse effects were observed on the pre-sealed plates.

The sealer itself gives good protection to the unused wells. Both Acetate and Mylar sealers are simple to use and provide a cheap and effective means of protecting an ELISPOT plate for multi-use purposes.

These procedures were replicated using cells and antigens in order for a full assessment to be made.

### EXAMPLE 4

### Recycling of Reagents for Use in an ELISPOT Assay

1. Peptide Pools (1 and 2) and Positive Control Reagent

Vials of peptide pools 1 and 2 and the Positive Control Reagent (PCR) were subjected to multi-use procedures in order to simulate the execution of an ELISPOT assay in series on recycled plates.

Peptide Pools 1 and 2 each comprise of a pool of peptides encoded by the RD1 region of the M. tuberculosis genome (Ewer, et al. Comparison of T-cell-based assay with tuberculin skin test for diagnosis of Mycobacterium tuberculosis infection in a school tuberculosis outbreak. The Lancet 2003; 361: 1168-1173.) Positive Control Reagent (PCR) is a 1 μg/ml mixture of the cellular mitogen PHA in cell culture medium (AIM-V).

Vials of Pool 1 and Pool 2 peptides, and Positive Control Reagent were opened and 50 μl of each solution was removed and discarded. The vials were then resealed and placed at 2-8° C. or 37° C. overnight. On days two and three the vials were removed from storage and on each occasion a further 50 μl of solution removed. The vials were then returned to their original storage conditions.

Pooled PBMC from five human donors were stored in liquid nitrogen, then thawed and used to assess the performance of the cycled reagents in an ELISPOT assay.

PBMC were thawed and washed in AIM-V media (GIBCO). Counting of viable PBMC was conducted using the trypan blue dye exclusion method as described in Cell Proliferation and Apoptosis (D. Hughes and H. Mehmet, 2003)

Reagents from the cycling procedures were added to a pre-coated 96 well microtiter plate in a typical ELISPOT procedure as described above.

Control wells contained fresh (uncycled) reagents. Negative control wells contained PBMC in the absence of Peptide Pools 1 or 2, or PCR.

TABLE 3
Spot counts (SFCs) generated as a result of the use
of Multi-use reagents stored at two temperatures versus
control reagents
Results

| | PCR | PCR | PCR | Pool 1 | Pool 1 | Pool 2 | Pool 2 |
|---|---|---|---|---|---|---|---|
| | 2-8° C. | 37° C. | Fresh | 2-8° C. | 37° C. | 2-8° C. | 37° C. |
| Mean | 30.5 | 33.5 | 8.25 | 1 | 1.5 | 3 | 3 |
| SFCs | | | | | | | |
| SD | 10.61 | 0.71 | 2.75 | 0.00 | 0.71 | 1.41 | 0.00 |
| % CV | 54.75 | 2.11 | 33.38 | 0.00 | 47.14 | 47.14 | 0.00 |

2. Conjugate Reagent

Vials of Conjugate Reagent were removed from storage and diluted 1:200 in PBS. After 1 hour the tube containing the diluted Conjugate Reagent was placed at 2-8° C. The original Conjugate Reagent vial was also placed back at 2-8° C. This cycle was repeated at 24 hour intervals up to four days. All diluted Conjugate Reagent solutions and the original stock solution were tested by running an Interferon Gamma assay as described in Example 3.

TABLE 4
Effect of Multi-use secondary conjugate on an ELISPOT plate containing various
concentrations of Interferon Gamma.
Results

| IFNγ | Day 1 | | Day 2 | | Day 3 | | Day 4 | | Fresh | |
|---|---|---|---|---|---|---|---|---|---|---|
| (ng/ml) | Mean | SD | Mean | SD | Mean | SD | Mean | SD | Mean | SD |
| 30 | 45.5 | 3.54 | 47.5 | 0.71 | 48.5 | 2.12 | 44.5 | 0.71 | 46.5 | 0.71 |
| 15 | 29 | 1.41 | 32 | 0.00 | 31.5 | 0.71 | 27.5 | 0.71 | 29 | 0.00 |
| 7.5 | 20.5 | 0.71 | 16 | 1.41 | 15 | 1.41 | 12 | 1.41 | 13.5 | 3.54 |
| 2.5 | 14.5 | 4.95 | 13.5 | 0.71 | 13 | 1.41 | 9.5 | 0.71 | 11 | 1.41 |
| 0.5 | 15.5 | 3.54 | 14.5 | 0.71 | 12.5 | 0.71 | 10 | 0.00 | 11 | 1.41 |

Using a Student T-Test there is no significant difference (a = 0.05) between the results obtained at all points. Mean represents % saturation of the test wells.

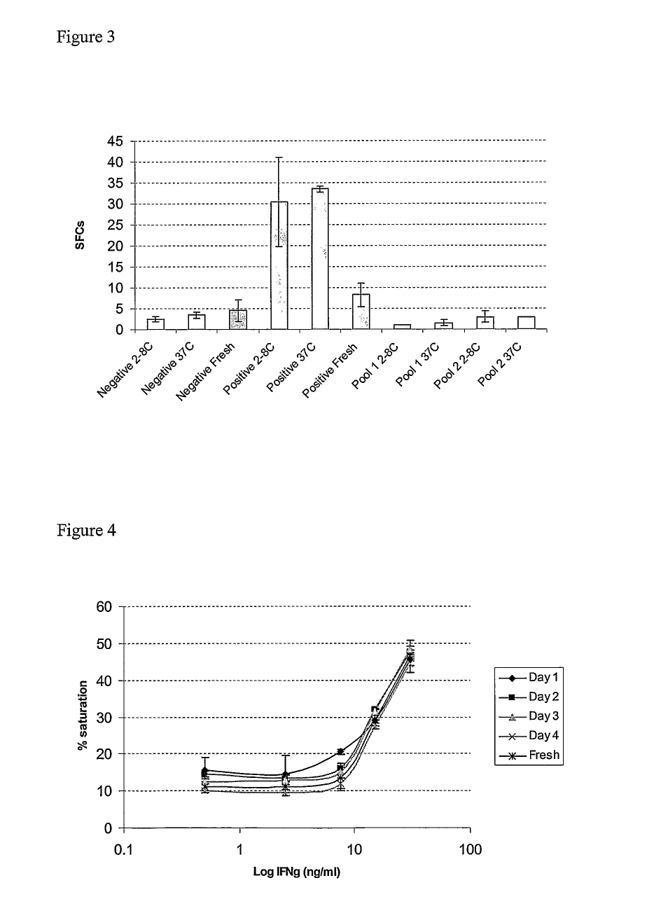

These results show the reagents used in the assay to be robust. (Tables 3 and 4. FIGS. 3 and 4.)

No difference in the spot count was seen in the negative controls, Pool 1 and Pool 2 Peptide results under all conditions. After visual inspection no contamination or discolouration was observed in any vial or reagent. The “fresh positive” sample shows a low spot count as the wells were fully saturated, thus the reader was unable to differentiate defined spots. From these results it is concluded that the reagents are robust under the recycling conditions applied.

### EXAMPLE 5

### Recycling of Reagents and Plates for Use in an ELISPOT Assay Using Donor PBMC

Microtiter plates were sealed with acetate sealing strips. Each plate was then subjected to a cycling regime to simulate the process of performing ELISPOT assays ‘in series’ on multiple occasions. This plate incubation regime comprised overnight incubation (16-20 hours) at 37° C. in a humidified incubator in a 5% CO₂ atmosphere followed by storage at 2-8° C. for eight hours. This regime was repeated up to six times.

Microtiter plates were cycled for 3, 4 and 5 times prior to the actual ELISPOT assay.

The performance of the plates after cycling was measured using the ELISPOT assay protocol using human donor samples (n=3). The CEF Peptide Pool antigen (Mabtech, Stockholm, Sweden) was used at 2 μg/ml per peptide to stimulate activated T-Cells and the IFN-γ released measured in the standard assay. A positive control solution (PHA, MP Biomedicals) at 1 μg/ml was measured against an un-stimulated cell control in a standard ELISPOT assay as described above.

TABLE 5
Mean results expressed in % Saturation of test wells and SFCs
Donor 1

| | Negative | Positive Control | CEF |
|---|---|---|---|
| | (SFCs) | (% Saturation) | (SFCs) |
| 0 Cycles | 0 | 44.5 | 45 |
| 3 Cycles | 0 | 32 | 44.5 |
| 4 Cycles | 0 | 29 | 45 |
| 5 Cycles | 0 | 31.5 | 39.5 |

TABLE 6
Mean results expressed in % Saturation of test wells and SFCs
Donor 2

| | Negative | Positive Control | CEF |
|---|---|---|---|
| | (SFCs) | (% Saturation) | (SFCs) |
| 0 Cycles | 0.5 | 60 | 46 |
| 3 Cycles | 1 | 52 | 59 |
| 4 Cycles | 1 | 46 | 50 |
| 5 Cycles | 0 | 41.5 | 40.5 |

TABLE 7
Mean results expressed in % Saturation of test wells and SFCs.
Donor 3

| | Negative | Positive Control | CEF |
|---|---|---|---|
| | (SFCs) | (% Saturation) | (SFCs) |
| 0 Cycles | 0 | 71.5 | 68.5 |
| 3 Cycles | n/a | n/a | n/a |
| 4 Cycles | 0.5 | 61 | 68 |
| 5 Cycles | n/a | n/a | n/a |

n/a = no results gained at this time-point.

The results from the three donors tested show that the spot count observed in response to CEF (an antigen used to simulate a positive T Cell response) is consistent at 0, 3, 4 and 5 cycles. This response suggests that the ELISPOT plate can be re-used up to five times and that an Acetate plate sealer provides adequate protection for the unused wells.

The positive control response from these three donors became weaker with increased cycling. It was observed that at the 5 cycle point the response had visually diminished. This observation was confirmed by the % saturation levels. The decrease in saturation was clear on all three donor samples suggesting this is an effect of the plate cycling process. It is likely that repeated cycling is causing a proportion of the coating antibody to become denatured. This may only be visible when saturation levels on the ELISPOT well are high (for example in the case of Interferon Gamma saturation caused by the Positive control solution). After five cycles this decrease in spot intensity did not affect the spot count.

These data support the recycling of the sealed ELISPOT plates up to four times.

### EXAMPLE 6

### Extended Use of ELISPOT Plates

Materials and Methods

White Cell Preparation

CD4 T cell clones that are known to respond to Mtb antigens were used as a source of white cells in this assay, with the T cell line D481 F4 responding to Peptide pool 1 and the T cell line D481 B9, D481 G7 and D454 E12 responding to Peptide pool 2 (obtained from D. M. Lewinsohn, Oregon Health & Science University, unpublished data). Antigen-presenting cells were provided by using autologous lymphocyte clonal lines (LCL) (also obtained from D. M. Lewinsohn, Oregon Health & Science University). The T cell clones and autologous LCLs were thawed quickly at 37° C. and 1 ml of pre-warmed (37° C.) GIBCO™ AIM-V™ cell culture medium (Invitrogen—product code 31035-025) was added dropwise to the cells. The volume was made up to 10 ml with AIM-V and centrifuged at 600×g for 7 minutes. The cell pellet was resuspended in 10 ml of fresh warm medium and centrifuged at 350×g for 7 minutes.

Viability Counting

Cell pellets were resuspended in 1 ml AIM-V; 10 μl was removed and diluted 5 fold in 0.4% (w/v) trypan blue; 10 μl of diluted cells was placed on the haemocytometer and viable cells counted with an inverted microscope. Cells were diluted to the appropriate cell number in AIM-V (10,000 TB cell clones per 100 μl and 20,000 LCL cells per 50 μl).

T SPOT Assay

The T SPOT-TB assay was performed according to the manufacturers instructions (Catalogue number: TB.200, Oxford Immunotec) with the exception that the assay required the addition of 50 μl of LCL (20,000 cells) and 100 μl T cell clones (10,000 cells) per well, as required, to the supplied 96 well membrane bottomed plate, pre-coated with anti-IFN-γ antibodies. Three T SPOT-TB plates were used in the short term study and two plates were used in the long term study, with each plate containing four replicates of each cell line per cycle (n=12 for short term study and n=8 for long term study).

The wells contained either Peptide pool 1 or Peptide pool 2 as appropriate. Plates were incubated for 16-20 hours at 37° C. with 5% CO₂. Wells were washed with PBS and developed with the supplied anti-IFN-γ antibody conjugate and enzyme substrate to reveal the presence of captured IFN-γ. Each spot records the footprint of one specific antigen reactive T cell.

Reuse of Plates

T SPOT plates were subjected to four rounds of the T SPOT-TB assay over a period of either ten days or two months. The assays were performed on days 1, 3, 8 and 10 for the short term study and on weeks 1, 2, 7 and 8 for the longer term study. In both studies Titer-Tops adhesive film (Fisher Scientific) was used to avoid contamination of unused wells. T SPOT plates were stored at 2-8° C. between assays.

Results

SFC counts for the T cell lines were compared when plates were subjected to four rounds of T SPOT-TB assays over a period of either ten days or two months. The T cell line D481 F4 responded to Peptide pool 1 and the T cell line D481 B9, D481 G7 and D454 E12 responded to Peptide pool 2.

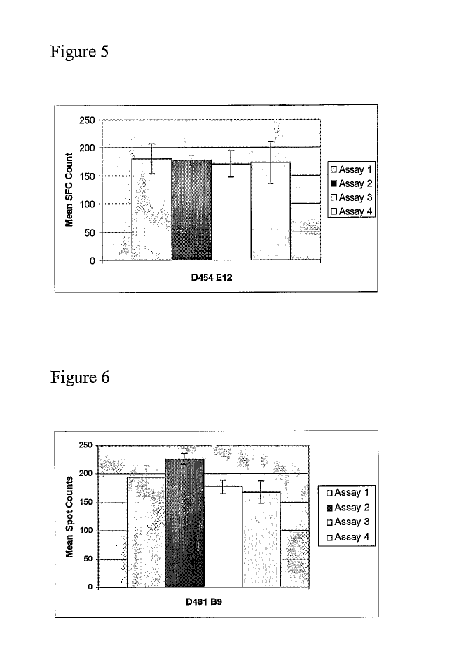

The results for sample D481 F4, D481 B9, D481 G7 and D454 E12 give a strong specific response to Mtb antigens. The SFC counts for D454 E12, for example, were consistent across all four assays in the short term study (FIG. 5) and when the SFC counts for assays 2, 3 and 4 were compared to assay 1 using the 2-sample t-test, assuming equal variances no significant difference (p≧0.05) was found across the four assays (Table 8). Equivalent results were found for D481 F4, D481 B9 and D481 G7 (data not shown).

TABLE 8
Statistical analysis of SFC counts for D454
E12 across four assays (short term study).

| | Assay | Assay | Assay | Assay |
|---|---|---|---|---|
| | 1 | 2 | 3 | 4 |
| P-value | — | 0.740 | 0.430 | 0.576 |

In the long term study similar results were observed across all four assays. The SFC counts for D481 B9, for example, show no particular trend across the four assays (FIG. 6).

The results show that consistent results can be obtained with T SPOT-TB across four assays over both a ten day and a two month period. This is presented, as an example, for T cell clone D454 E12 and D481 B9. Equivalent results were obtained for all cell lines that were tested in both studies.

1. An immunoassay comprising:—
(a) providing an assay plate having a plurality of recesses, the recesses having solid supports therein having antibody bound thereto;
(b) carrying out an assay in one or more of the recesses but not in all of the recesses having antibody bound thereto;
(c) subsequently carrying out an assay in one or more of the recesses that were not used in step (b).

2. An assay according to claim 1 further comprising:
(d) repeating step (c) one or more times to carry out an assay in one or more of the recesses that were not used in the assays of (b) and (c).

3. An assay according to claim 1 wherein prior to step (b) one or more of the recesses are covered, step (b) being carried out in recesses which are not so covered, and one or more of said covered recesses being uncovered in order to carry out the assay of step (c).

4. An assay according to claim 3 wherein said cover seals said recesses.

5. An assay according to claim 4 wherein said cover comprises a sealing membrane.

6. An assay according to claim 1, wherein the assay of step (b) and/or (c) comprises incubating said plate at a temperature of 25° C. or more for a period of 1 hour or more.

7. An assay according to claim 6, wherein the assay of step (b) and/or (c) comprises incubating said plate at a temperature between 25° C. to 60° C. for a period between 1 to 24 hours.

8. An assay according to claim 1 wherein the assay plate is stored between each assay of step (b) and/or (c).

9. An assay according to claim 8 wherein the assay plate is stored up to 7 days between each assay of step (b) and/or (c).

10. An assay according to claim 9 wherein the assay plate is stored up to 2 days between each assay of step (b) and/or (c).

11. An assay according to claim 8 wherein the assay plate is stored between −5 to 1O° C.

12. An assay according to claim 11 wherein the assay plate is stored between 2 to 8° C.

13. An assay according to claim 1 wherein the assay plate comprises a microtiter plate.

14. An assay according to claim 1 wherein the solid support is a solid permeable support.

15. An assay according to claim 14 wherein the solid supports comprise a PVDF membrane or a nitrocellulose membrane.

16. An assay according to claim 1 wherein each assay plate is used to conduct up to 5 separate assays.

17. An assay according to claim 1 wherein the assay is a cell-based assay.

18. An assay according to claim 17 wherein the assay is an ELISPOT assay.
